(19) United States
(12) Plant Patent Application Publication
(10) Pub. No.: US 2002/0002718 P1
(43) Pub. Date: Jan. 3, 2002

(54) Camellia plant named 'N# 9205'

(76) Inventor: Julius Nucio, Sunset Beach, CA (US)

Correspondence Address: Christie Parker & Hale LLP, Post Office Box, Pasadena, CA, 91109-7068, US

(21) Appl. No.: 09/564,250

(22) Filed: May 4, 2000

(51) Int. Cl. A01H 5/00

(52) U.S. Cl. PLT/245

(57) ABSTRACT

A new plant variety of Camellia with attractive red flowers.

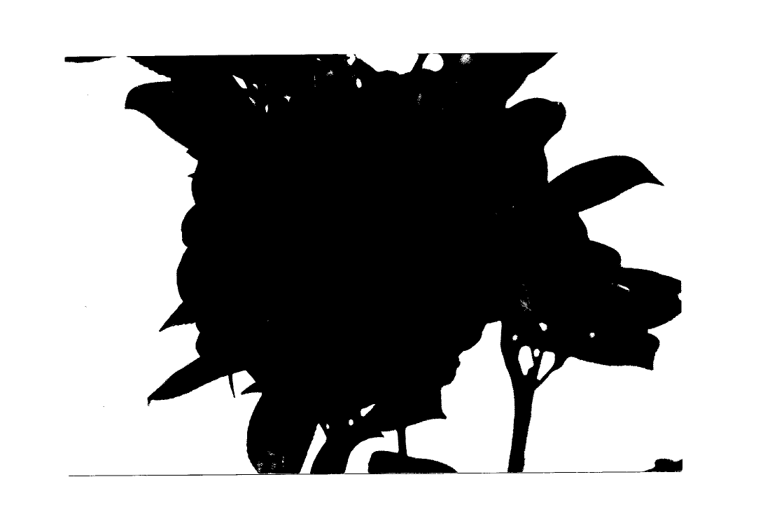

### BACKGROUND OF THE INVENTION

The present invention relates to a new and distinct variety of Camellia and was discovered as a seedling in a controlled planting of Camellia japonica in Altadena, Calif. The varietal denomination of the new variety is ‘N#9205’.

The genus Camellia is included in the family Theaceae which comprises about 25 genera of shrubs and trees, mostly tropical and subtropical, growing in the northern and southern hemispheres. Camellia comprises over 80 species of evergreen shrubs or trees, many of which possess desirable ornamental characteristics.

### SUMMARY OF THE INVENTION

The new variety produces attractive red flowers. Asexual reproduction of the new variety by grafting and stem cuttings performed in Altadena, Calif. has confirmed that the distinctive characteristics of the new variety are stable and transmitted to succeeding generations.

### BRIEF DESCRIPTION OF ILLUSTRATIONS

The accompanying illustration shows a flower of the new cultivar in a photo illustration as true to color as is reasonably possible to make in an illustration of this character.

### DESCRIPTION OF THE NEW VARIETY

‘N#9205’ has not been observed under all possible environmental, cultural, and light conditions. The following observations and descriptions are of plants grown in Altadena, Calif. In the description, color references are to The Royal Horticultural Society Colour Chart and terminology used in the color descriptions herein refers to plate numbers in this color chart. Phenotypic expression may vary with light intensity, cultural, and environmental conditions.

Plant:

Form.—Upright, fairly compact.

Growth.—Medium, up to about 4 feet.

Leaves.—Medium, japonica-sized leaves; upperside medium green near 147A to 147B; underside lighter near 147C.

Shape.—Same as Camellia japonica.

Wood.—New shoots are of amber color, near 23C to 23B.

Bark.—Typical of japonica; light green when immature, turning to brown then aging to grey.

Flower:

Size.—Large to very large.

Flower.—Double; medium red, occasionally showing lavender tones near margin of petals; about 55-70 petals; near 46B.

Fragrance.—None.

Reproductive Organs.—Plant is not fertile and has no stamen or stigma, produces no pollen and hips.

I claim:

1. A new plant of Camellia substantially as shown and described.
